(19) United States
(12) Patent Application Publication
(10) Pub. No.: US 2004/0115823 A1
(43) Pub. Date: Jun. 17, 2004

(54) METHOD FOR IMPROVEMENT OF ENVIRONMENTAL STABILITY AND SELECTIVITY OF MATERIALS FOR SENSORS

(76) Inventor: Radislav Alexandrovich POTYRAILO, Niskayuna, NY (US)

Correspondence Address: FOLEY AND LARDNER, SUITE 500, 3000 K STREET NW, WASHINGTON, DC, 20007, US

(21) Appl. No.: 10/248,038

(22) Filed: Dec. 12, 2002

(51) Int. Cl. G01N 33/00

(52) U.S. Cl. 436/124; 422/68.1; 422/69; 422/50; 436/176; 436/183

(57) ABSTRACT

A protective overlayer for a sensor array sensing surface includes an amorphous fluoropolymer overlayer overlaid on different sensing films, the amorphous fluoropolymer being resistant to common organic solvents, alkaline and acid solutions.

### BACKGROUND OF INVENTION

This invention relates to materials and sensor devices for the real-time quantitation of volatile compounds in fluids or the like.

Sensor films or coatings are often needed to improve and enhance the detection ability of sensors. For example, in optical detection, a large variety of analytes are not optically active in the spectral range where the measurements can be potentially be done using available cost-effective instrumentation. Thus, numerous reagents (colorimetric, fluorescent, chemoluminescent, etc.) are used when a reagent is incorporated into a polymer film and is changing its optical property upon exposure to analytes.

A variety of materials can be applied as sensor coatings and analyte-coating interactions can be detected using different sensing techniques. Vapor-sorbing materials are used in piezoelectric, optical, and other types of sensors. Unfortunately, typical sorbing polymer coatings easily loose their performance (stability and sensitivity) upon interactions with organic solvents, highly alkaline solutions and the like materials. This, of course, leads to loss of sensor efficiency and/or malfunction.

PCT Published Application WO 01/67086, resulting from U.S. patent application Ser. No. 09/519,891, filed Mar. 6, 2000, both incorporated herein in their entirety, disclose an amorphous fluoropolymer sensing layer coated on the array substrate. The amorphous fluoropolymer sensing layer is generally mechanically and chemically stable upon exposure to non-polar solvents, and provides an acceptable frequency shift of about 24 Hz when used in a quartz microbalance sensor upon exposure to differing analyte concentration. However, it would be desirable to obtain a sensor with a higher sensitivity to analyte ratio and concentration.

U.S. Published application Ser. No. 2002/0,173,040 A1, published on Nov. 21, 2002 and incorporated by reference herein in its entirety, discloses a method of testing barrier film performance. In this method, a single sensing film is covered by an amorphous fluoropolymer layer. A plurality of different barrier film segments are located over the fluoropolymer layer. The barrier film segments are exposed to a known fluid of a known concentration and ratio of fluid components. The resistance of the barrier film segments to the fluid is then determined.

### SUMMARY OF INVENTION

A first aspect of the invention resides in a sensor array, comprising a plurality of different sensor films, and a sensor-protecting overlayer located over the plurality of sensing films, comprising a film which is in contact with the sensing films, and which has characteristics which make it permeable to analytes of interest to be measured with the sensing films without substantially altering a response of the sensing film upon exposure of the sensor to the analytes (i.e. altering a response of the sensing film by less than 20%, preferably by less than 10%).

A second aspect of the invention resides in a sensor array comprising a substrate, a plurality of different sensing films on the substrate, and an amorphous fluoropolymer protective overlayer overlaid on the sensor films.

A third aspect of the invention resides in a method of determining a nature and concentration of at least one analyte component comprising: providing a sensor comprising a substrate, at least one sensing layer and an amorphous fluoropolymer protective layer, exposing the sensor to at least one analyte, and determining at least one of the nature and the concentration of at least one analyte component.

A fourth aspect of the invention resides in a chemical sensor system for analysis of at least one analyte of interest comprising: a chemical sensor having a sensing layer responsive to the at least one analyte of interest; a protective layer deposited onto the sensing film that protects the sensing layer from being degraded by a fluid in a measured sample containing the at least one analyte, wherein degradation comprises a loss of the integrity of the sensing film due to dissolution or adhesion loss and wherein the sensor detection parameters of the sensing film are unaltered by the protective layer; and a sensor signal multivariate analysis system where multivariate analysis is performed by analyzing at least two responses from the chemical sensor, wherein the at least two responses are a combination of time response points during sample measurement.

A fifth aspect of the invention resides in an optical sensor system for analysis of at least one analyte of interest comprising: an optical sensor having a sensing layer responsive to the at least one analyte of interest; a protective layer deposited onto the sensing layer that protects the sensing layer from being degraded by a fluid in a measured sample containing the analyte of interest, wherein degradation comprises loss of the integrity of the sensing film due to dissolution or adhesion loss and wherein the optical detection parameters of the sensing layer are unaltered by the protective layer; and a sensor signal multivariate analysis system wherein multivariate analysis is performed by analyzing at least two responses from the optical sensor, and wherein at least two responses are the combination of time response points during sample measurement.

Yet another aspect of the invention resides in an acoustic-wave sensor system for analysis of at least one analyte of interest comprising: an acoustic-wave sensor having a sensing layer responsive to at least one analyte of interest; a protective layer deposited onto the sensing layer that protects the sensing layer from being degraded by a fluid in a measured sample containing the at least one analyte, wherein degradation comprises loss of the integrity of the sensing film due to dissolution or adhesion loss and wherein the acoustic-wave detection parameters of the sensing film are unaltered by the protective layer; and a sensor signal multivariate analysis system where multivariate analysis is performed by analyzing at least two responses from the acoustic-wave sensor, wherein at least two responses are a combination of time response points during sample measurement.

### BRIEF DESCRIPTION OF DRAWINGS

FIG. 1 is a graphical comparison of dynamic response of sensor regions with and without a protective overlayer for an acoustic wave sensor upon introduction of a selected fluid.

FIG. 2 is a graphical comparison of dynamic response of sensor regions with and without a protective overlayer for an acoustic wave sensor upon the purging of a selected fluid.

FIG. 3 is a graphical comparison of dynamic response of three optical sensors with a protective overlayer and three optical sensors without a protective overlayer upon exposure to a selected fluid and a blank gas.

FIG. 4 is a schematic diagram showing how the sensing layer of a sensor can be provided with a protective overlayer and used as part of a multivariate analysis system.

### DETAILED DESCRIPTION OF THE INVENTION

Environmental stability of sensor materials is achieved through the use of amorphous fluoropolymer materials as a protective overlayer for sensor arrays. This protective overlayer is applied over a chemically sensitive/susceptible film and/or films, referred to as sensing film or films. These sensing films preferably comprise polymer or other suitable materials which are deposited onto or form part of the sensor/transducer, which is referred to as a substrate. The substrate may comprise any material, such as polymer, ceramic, glass, quartz, metal or semiconductor, that is suitable for use in a particular type of sensor array. The overlayer protects the chemically sensitive film or films from one or more of dissolution, removal or damage by solvents/corrosive materials and thus prolong the working life of the sensor array.

The protective fluoropolymer overlayer material preferably comprises amorphous copolymers of perfluoro-2,2-dimethyl-1,3-dioxole. The protective overlayers may comprise one amorphous fluoropolymer or a combination of two or more such materials at least one of which is a fluoropolymer.

Unlike other types of sorbing materials, protective overlayer films made of amorphous fluoropolymers are extremely stable to even nonpolar solvents that completely dissolve conventional polymeric films coated onto sensor sensing surfaces. Thus, highly robust sensors can be built by disposing protective overlayers on top of sensing films which exhibit chemical sensitivity and are susceptible to damage by chemicals/solvents.

Preferably, a plurality of different sensing films are located in spaced apart locations on the sensor substrate. The use of different sensing films (i.e., sensing film segments) of different composition allows the sensor array to be used to determine the nature and/or concentration of the analyte components to which the sensor array is exposed to. For example, different sensing films can be located at different parts of the sensor array to determine which and how many components are present in the analyte mixture and/or the level or concentration of each component in the analyte mixture. Preferably, the sensing films are much more sensitive to the nature and/or concentration of the analyte than the protective overlayer. The formation of a protective overlayer over a plurality of sensing films provides a sensor array which is mechanically and chemically stable upon exposure to various analytes while providing a higher response rate to analyte component nature and/or concentration than a sensor in which the amorphous fluoropolymer layer is used as the sensing layer. The protective overlayer may be a single continuous layer located over the plurality of different sensing films or a plurality of segments of protective overlayer located over the plurality of sensing film segments.

The sorption properties of amorphous fluoropolymers to analytes of different molecular weight and nature vary as a function of fluoropolymer composition. This feature makes the overlayer material adjustable and suitable for applications in different sensor arrays. By way of example, by adjusting the ratio of one polymer component to another in the fluoropolymer composition, the sorption characteristics of the protective overlayer can be matched or rendered proximate to the sorption characteristics of the sensor sensing layer and thus avoid any reduction in sensor sensitivity.

As will be understood, the amorphous fluoropolymer films are applicable at least to sensors of the type which are based on the interactions of a chemically sensitive film with an environment, and detection of a variation of certain film property or properties as the function of analyte concentration or concentrations of multiple analytes in a mixture. Preferably the amorphous fluoropolymer films are used as overlayers which are disposed on top of chemically sensitive materials that form transducer elements of the sensor and thus protect the sensor from damage while permitting the necessary chemical interaction to take place.

The sensing film property which is used in the above type of sensor can comprise a change in mass of the film, its viscoelastic property, or other mechanical property, as well as dielectric and optical properties. The optical properties can be altered with an analyte partitioned into the sensor film and can be monitored as the change in the absorbance, scattering, refractive index, luminescence, or another property of the sensor film. Also, a chemically sensitive dye can be incorporated into the bulk of the polymer sensing film or dye molecules can be directly attached to the polymer molecules of the sensing film. Changes of optical properties of the dye can be related to the variation of the chemical environment around the sensor.

Preferred examples of protective overlayer materials for applications in chemical sensors, are random copolymers of tetrafluoroethylene (TFE) and perfluoro-2,2-dimethyl-1,3-dioxole (PDD) sold under the trademark TEFLON AF®. For film deposition, amorphous fluoropolymers can be dissolved in perfluoro(2-butyl tetrahydrofuran) and thin films from polymer solution can be deposited onto the surface of the sensor by dip-coating, spin-coating, spraying, brushing, or by applying other techniques. Variation of the chemical sorption property of thin overlayer films made of these materials is achieved by the alteration of the TFE/PDD ratio. Materials with different TFE/PDD ratio are useful for applications in sensor arrays.

Other related types of amorphous fluoropolymers can be also used as protective overlayers in chemical sensors. These materials can be amorphous terpolymers of PDD with TFE and another comonomer. Other variations of protective overlayer materials of the same family of amorphous fluoropolymers are dipolymers and terpolymers (collectively, copolymers) of PDD with comonomers, which include certain perfluoroolefins and perfluoro(alkyl vinyl ethers). Further variations of protective overlayer materials useful for chemical sensors are amorphous homopolymers and copolymers containing repeating cyclic structures formed during the cyclic polymerization of perfluoro(butenyl vinyl ether) (PBVE). All these types of materials exhibit an excellent resistance to common organic solvents, alkaline and acid solutions.

When different sensing film materials are used as chemically sensitive coatings on individual transducers in sensor arrays, selective analyte detection in complex mixtures can be achieved by analyzing the response pattern of a sensor array by means of known pattern recognition techniques. The use of sensing film coatings comprised of two or more layers on individual transducers in sensor arrays can change response pattern in the array. This change provides an additional possibility to creation more diverse sensor arrays.

The optical properties of a sensing film can be altered with an analyte partitioned into the sensor film and can be monitored as the change in the absorbance, scattering, refractive index, luminescence, or another property of the sensor film. The optical spectrum may be monitored at one wavelength for univariate analysis, or at more than one wavelength for multivariate analysis. In an embodiment, the optical characteristics of the sample are analyzed using statistical techniques. For example, the optical characteristics of the sample may be analyzed using univariate linear regression calibration methods (see e.g. H. Mark and J. Workman, Statistics in Spectroscopy: Academic Press: San Diego, Calif., pp. 263-276 (1991 ); and J. C. Miller and J. N. Miller, Statistics for Analytical Chemistry, Ellis Horwood, New York, N.Y., pp. 101-139 (1993)). Univariate calibration models may be derived which provide quantitative prediction of analyte concentration in a sample based on optical measurements at one wavelength. Alternatively, univariate calibration models may be derived which provide quantitative prediction of analyte concentration in a sample based on optical measurements at one wavelength. Also, different wavelengths in a spectrum can be used to relate to the concentrations of different analytes in the sample.

Where the optical spectrum comprises several wavelengths or an entire spectrum over a certain range, the optical characteristics of the sensing film may be determined using multivariate calibration algorithms such as Partial Least Squares Regression (PLS), Principal Components Regression (PCR), and the like (see e.g. Beebe, K. R. et al., Chemometrics: A Practical Guide; Wiley, New York, N.Y., pp. 183-339 (1998)). Given a large enough span of calibration samples, multivariate calibration models are generally more robust than univariate models due to enhanced outlier detection capabilities and increased tolerance toward slight shifting in peak position or band shape. Also, multivariate calibration models allow for measurement of more than one variable or component of interest with a single sensing film. PLS models correlate the sources of variation in the spectral data with sources of variation in the sample. Preferably, the PLS model is validated by statistical techniques. Such statistical techniques include, but are not limited to, leave one out cross-validation, venetian blinds, and random subsets (see e.g. Beebe, K. R., et al., Chemometrics: A Practical Guide, Wiley, New York, N.Y. (1998)).

In the acoustic-wave detection, similar univariate and multivariate responses can be used from a single sensor or a group of sensors arranged in an array. For example, a multivariate response from a single sensor can be obtained if several outputs from a sensor are monitored. These can include fundamental frequency, attenuation, phase change, and others at a steady state mode of sensor response or as monitored dynamically over time of a measurement. When a group of sensors coated with different films are used, their combined response is also analyzed using multivariate analysis tools.

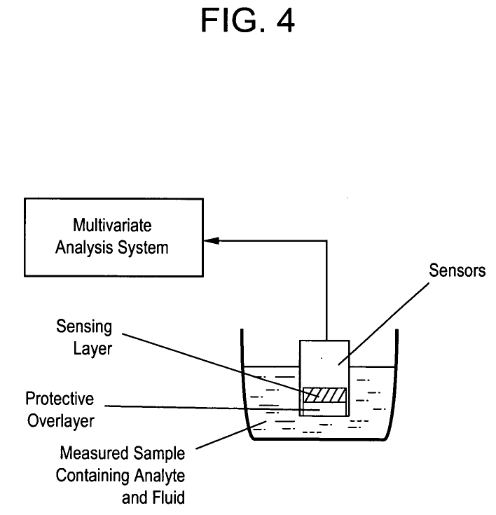

As will be recognized by those of ordinary skill in the art, all or part of the steps in the analysis of response of sensing films to analytes may be coded or otherwise written in computer software, in a variety of computer languages including, but not limited to, C, C++, Pascal, Fortran, Visual Basic®, Microsoft Excel, MATLAB®, Mathematica®, Java, and the like. Accordingly, additional aspects of the present invention include computer software for performing one or more of the method steps set forth herein. The software code may be compiled and stored in executable form on computer readable media as, for example, computer ROM, floppy disk, optical disk, hard disks, CD ROM, or the like. Both the hardware and software are included in the Multivariate Analysis System shown in FIG. 4.

### EXAMPLES

1) Acoustic Wave Sensors. For experimental demonstration of performance of amorphous fluoropolymers, TEFLON AF 2400 was used as the protective overlayer. The polymer was applied as a thin film onto the surface of a chemically sensitive film on a thickness shear mode (TSM) sensor. The change in polymer mass of the sensor film was monitored upon exposure to analyte vapors. As the sensor substrate, an AT-cut quartz crystal with gold electrodes was used. These crystals oscillate in the thickness-shear mode with a fundamental frequency of 10 MHz. For film deposition, the amorphous fluoropolymer TEFLON AF 2400 was dissolved in perfluoro(2-butyl tetrahydrofuran) (fluorinert, electronic liquid FC-75, 3M Company). The polymer solution was applied to both surfaces of the crystal and dried. Prior deposition of the protective overlayer, a chemically sensitive (Siltem 2000) polymer film was deposited The use of this polymer as chemically sensitive vapor sorbing film is disclosed in U.S. Pat. No. 6,357,278 B1 incorporated by reference. The polymer used to form this chemically sensitive layer was dissolved in chloroform, the solution was applied to both surfaces of the crystal, and dried.

Crystals with and without the overlayer were arranged in an array and exposed to different concentrations of solvent vapors. The resonant oscillation frequency of the array of transducers was monitored using 225-MHz Universal Counters (model HP 53132A, Hewlett Packard, Santa Clara, Calif.) as a function of time. Data acquisition was performed with a laptop PC using a program written in LabVIEW (National Instruments, Austin, Tex.).

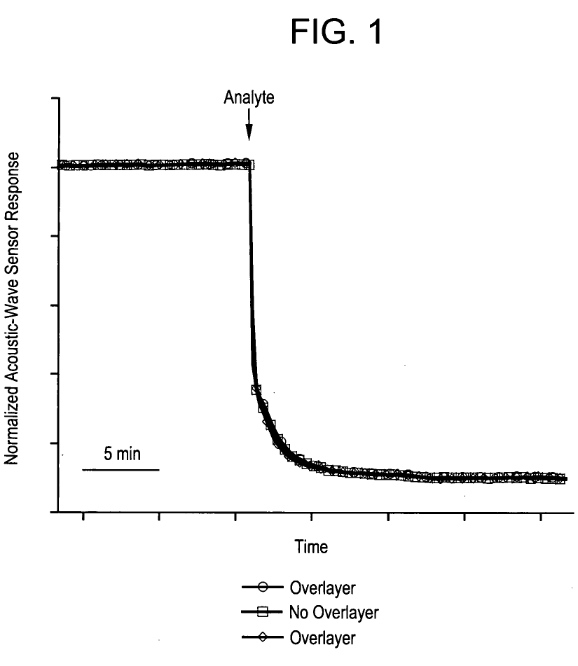

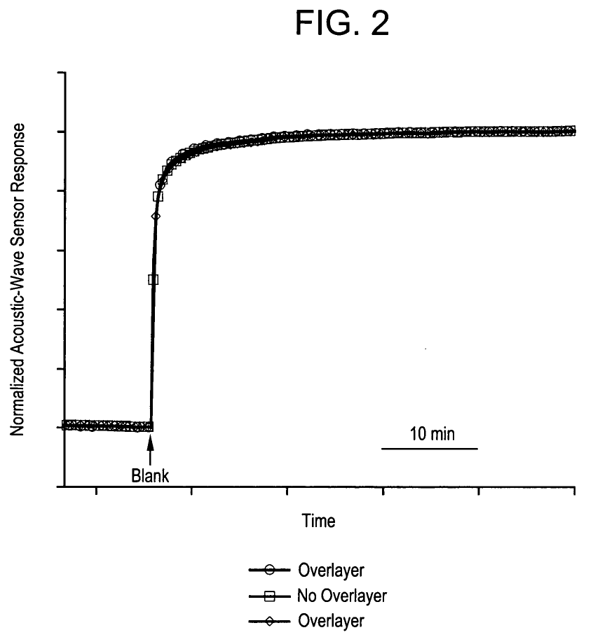

Upon exposure of the TSM sensor array to varying concentrations of vapors, the signal change was recorded. Sensor response was observed to be completely reversible and rapid (less than a minute) indicating the attractive rapid sorption/desorption characteristics of the material composition. Comparison of dynamic response of sensors without and with the protective overlayer demonstrated that the overlayer does not significantly increase the response time of the sensor structure upon their exposure to vapors of organic solvents at concentrations of 10-100 ppm. A typical plot of the dynamic response of sensors with and without the protective overlayer upon introduction of toluene vapor is presented in FIG. 1. A typical plot of the dynamic response of sensors with and without the protective overlayer upon purging toluene vapor with a blank gas (nitrogen) is presented in FIG. 2.

2) Optical Sensors. The response of an optical sensor based on a reagent-doped polymeric material was evaluated. An analyte sensitive dye was dissolved in a polymer solution in an organic solvent. Sensing film deposition was performed using a 20-microliter volume of the film formulation and pipetting the formulation onto a PET sheet and evaporating the solvents from the film. As an example of a chemically sensitive film for an optical sensor, nile red fluorophore was incorporated at a 1-mM concentration in 5% w/w solution of perfluorosulfonic acid-PTFE copolymer.

This sensing film was analyzed before and after exposure to moist air, with a relative humidity of about 80%. Luminescence measurements were performed on a setup which included a white light source (450-W Xe arc lamp, SLM Instruments, Inc., Urbana, Ill., Model FP-024), a monochromator for selection of the excitation wavelength (SLM Instruments, Inc., Model FP-092), and a portable spectrofluorometer (Ocean Optics, Inc., Dunedin, Fla., Model ST2000).

The spectrofluorometer was equipped with a 200-μm slit, 600-grooves/mm grating blazed at 400 nm and covering the spectral range from 250 to 800 nm with efficiency greater than 30%, and a linear CCD-array detector. Excitation light from the monochromator was focused into one of the arms of a “six-around-one” bifurcated fiber-optic reflection probe (Ocean Optics, Inc., Model R400-7-UV/VIS). Emission light was collected from a sample when the common end of the fiber-optic probe was positioned near the sample at a certain angle to minimize the amount of excitation light reflected from the sample back into the probe. The second arm of the probe was coupled to the spectrofluorometer. The sensor film coating was positioned in a flow-through cell with transparent windows for optical analysis of materials in the cell. The cell with the sensor film coating was positioned on an automated XY translation stage.

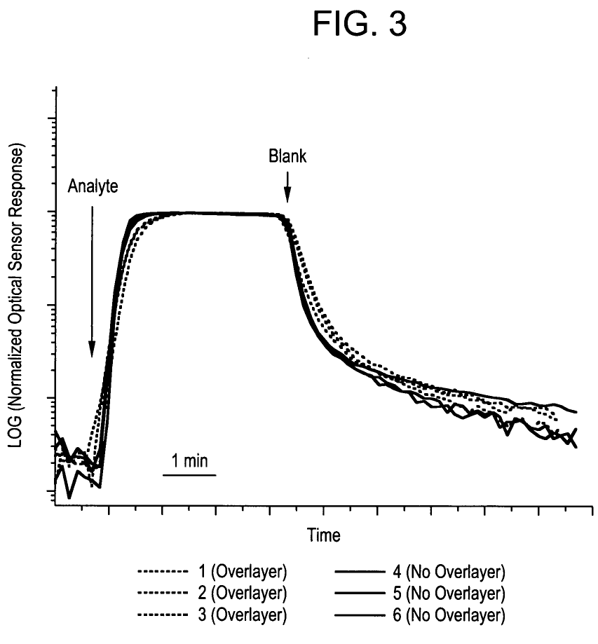

Three replicates of this sensing film formulation were coated with a solvent resistant protective overlayer. Comparison of dynamic response of sensor regions without and with the protective overlayer demonstrated that the overlayer does not significantly increase the response time of the sensor structure. A typical plot of the dynamic response of three sensor regions with the protective overlayer made of Teflon AF 2400 and three sensor regions without the protective overlayer is presented in FIG. 3.

### Environmental Stability

The environmentally-protective overlayer may be applied to any type of sensor to shield the sensing film or layer of the sensor. For example, the protective overlayer may be incorporated into optical sensors, acoustic wave sensors, chemical resistors, conductivity sensors, MEMS (Micro Electromechanical Systems) sensors, etc., in order to improve the accuracy of determining analyte of interest.

Unlike other types of polymer films, the protective overlayer films made of amorphous fluoropolymers are stable upon direct contact with a variety of common aggressive solvents such as those used for dissolving of conventional polymers (chloroform), as well as gasoline simulators (hexane/toluene mixture, 9:1 ratio), highly alkaline solutions (ammonium hydroxide) which decomposes RTV silicone films, and others. It was found experimentally that the protective overlayer films deposited onto the surface of a quartz crystal can be removed only by applying a highly specific solvent, perfluoro(2-butyl tetrahydrofuran) (fluorinert, electronic liquid FC-75, 3M Company). Thus, the protective overlayer is permeable to analytes of interest to be measured with the sensor film without altering a response of the sensor film upon exposure of the sensor to the analytes. Table 1 summarizes the environmental stability of the films.

TABLE 1
Environmental stability of amorphous fluoropolymer films.

| Solvent | Film performance |
|---|---|
| Hexane/toluene mixture, 9:1 ratio | Stable |
| Chloroform | Stable |
| Ammonium hydroxide | Stable |
| Methanol | Stable |
| Acetone | Stable |
| Perfluoro(2-butyl tetrahydrofuran | Dissolved |

It will be appreciated that even though the invention has been disclosed with only reference to a limited number of embodiments/examples, and that the protective overlayer which has been disclosed is well suited for protecting chemically susceptible sensor surfaces against the deleterious effects of common solvent, alkaline and acid solutions including those “common” solvents and solutions exemplified above. It will also be appreciated that the proceeding disclosure is for the purpose of illustration and that this description should not be deemed a limitation as to the scope of the invention. Given this disclosure various modifications, adaptations and alternatives will be readily apparent to the person of skill in the art to which the claimed subject matter pertains, or most closely pertains, without departing from the scope of the invention which is limited only by the appended claims.

1. A sensor array, comprising:
a plurality of different sensing films; and
a sensor-protecting overlayer located over the plurality of different sensing films, comprising a film which is in contact with the sensing films, and which has characteristics which make it permeable to analytes of interest to be measured with the sensing films without substantially altering a response of the sensing films upon exposure of the sensor to the analytes.

2. A sensor array of claim 1, wherein the overlayer comprises an amorphous fluoropolymer overlayer.

3. A sensor array as set forth in claim 2, wherein the sensor array comprises a chemical, optical or acoustic sensor array.

4. A sensor array as set forth in claim 2, wherein:
the sensing films are located on a sensor substrate;
the overlayer is in contact with the sensing films; and
the overlayer has characteristics which make it permeable to analytes of interest to be measured with the sensor without altering a response of the sensor upon exposure of the sensor to the analytes.

5. A sensor array as set forth in claim 2, wherein the amorphous fluoropolymer overlayer is formed of a mixture of tetrafluoroethylene (TFE) and perfluoro-2,2-dimethyl-1,3-dioxole (PDD).

6. A sensor array as set forth in claim 5, wherein a ratio of TFE to PDD is selected in accordance with sorption properties of the sensing films.

7. A sensor array as set forth in claim 2, wherein the amorphous fluoropolymer sorbing overlayer comprises amorphous terpolymers with a comonomer.

8. A sensor array as set forth in claim 2, wherein the amorphous terpolymers comprise terpolymers of TFE and PDD.

9. A sensor array as set forth in claim 2, wherein the amorphous fluoropolymer overlayer comprises dipolymers and terpolymers of PDD with at least one other comonomer.

10. A sensor array as set forth in claim 9, wherein the comonomer is selected from the group consisting of perfluoroolefins and perfluoro(alkyl vinyl ethers).

11. A sensor array as set forth in claim 2, wherein the amorphous fluoropolymer is selected from the group consisting of amorphous homopolymers and copolymers containing repeating cyclic structures formed during the cyclic polymerization of perfluoro(butenyl vinyl ether).

12. A method of determining nature and concentration of at least one analyte component, comprising:
providing a sensor comprising a substrate, at least one sensing layer and at least one amorphous fluoropolymer protective layer;
exposing the sensor to the at least one analyte; and
determining at least one of the nature and the concentration of at least one analyte component.

13. A method as set forth in claim 12, further comprising forming the at least one sensing film over a sensor sensing surface prior to forming the amorphous fluoropolymer overlayer.

14. A method as set forth in claim 12, wherein the amorphous fluoropolymer comprises a mixture of tetrafluoroethylene (TFE) and perfluoro-2,2-dimethyl-1,3-dioxole (PDD).

15. A method as set forth in claim 14, further comprising the steps of:
determining sorption characteristics of the at least one sensing film; and
selecting a ratio of TFE to PDD in accordance with the determined sorption characteristics.

16. A method as set forth in claim 12, further comprising the step of forming the amorphous fluoropolymer from terpolymers with a comonomer.

17. A method as set forth in claim 16, wherein the amorphous terpolymers comprise terpolymers of TFE and PDD.

18. A method as set forth in claim 12, further comprising the step of forming the amorphous fluoropolymers of dipolymers and terpolymers of PDD with at least one other comonomer.

19. A method as set forth in claim 18, further comprising the step of selecting the comonomer from the group consisting of perfluoroolefins and perfluoro(alkyl vinyl ethers).

20. A method as set forth in claim 12, further comprising the step of selecting the amorphous fluoropolymer from the group consisting of amorphous homopolymers and copolymers containing repeating cyclic structures formed during the cyclic polymerization of perfluoro(butenyl vinyl ether).

21. A method as set forth in claim 13, further comprising the step of dissolving the amorphous fluoropolymer in a solvent and depositing the amorphous fluoropolymer overlayer on the at least one sensing film using a selected technique.

22. A method as set forth in claim 21, wherein the overlayer is deposited using dip coating.

23. A method as set forth in claim 21, wherein the overlayer is deposited using spin-coating.

24. A method as set forth in claim 21, wherein the overlayer is deposited using spraying.

25. A method as set forth in claim 21, wherein the overlayer is deposited using brushing.

26. A method as set forth in claim 12, wherein the at least one sensing film comprises a plurality of different sensing films.

27. A chemical, optical or acoustic-wave sensor system for analysis of at least one analyte of interest comprising:
a chemical, optical or acoustic-wave sensor having a sensing film responsive to the at least one analyte of interest;
a protective layer deposited onto the sensing film that protects the sensing film from being degraded by a fluid in a measured sample containing the at least one analyte, wherein degradation comprises a loss of the integrity of the sensing film due to dissolution or adhesion loss and wherein the chemical, optical or acoustic-wave detection parameters of the sensing film are unaltered by the protective layer; and
a sensor signal multivariate analysis system where multivariate analysis is performed by analyzing at least two responses from the chemical, optical or acoustic-wave sensor, wherein the at least two responses are a combination of time response points during sample measurement.

28. A system as set forth in claim 27, wherein the sensor comprises a chemical sensor.

29. A system as set forth in claim 27, wherein the sensor comprises an optical sensor.

30. A system as set forth in claim 27, wherein the sensor comprises an acoustic-wave sensor.

31. A system as set forth in claim 27, wherein the protective layer comprises an amorphous fluoropolymer layer which has characteristics which make it permeable to analytes of interest to be measured with the sensing film without altering a response of the sensing film upon exposure of the sensor to the analytes.

32. A sensor array, comprising:
a substrate;
a plurality of different sensing films on the substrate; and
an amorphous fluoropolymer protective overlayer overlaid on the sensing films.

33. A sensor array as set forth in claim 32, wherein the amorphous fluoropolymer comprises a mixture of tetrafluoroethylene (TFE) and perfluoro-2,2-dimethyl-1,3-dioxole (PDD).

34. A sensor array as set forth in claim 33, wherein a ratio of TFE to PDD is selected in accordance with desired sorption properties of the sensing film.

35. A sensor array set forth in claim 32, wherein the protective overlayer is permeable to analytes of interest to be measured with the sensor without substantially altering a response of the sensor upon exposure of the sensor to the analytes.

36. A sensor array as set forth in claim 32, wherein the amorphous fluoropolymer comprises amorphous terpolymers with a comonomer.

37. A sensor array as set forth in claim 36, wherein the amorphous terpolymers comprise terpolymers of TFE and PDD.

38. A sensor array as set forth in claim 32, wherein the amorphous fluoropolymer comprise dipolymers and terpolymers of PDD with at least one other comonomer.

39. A sensor array as set forth in claim 38, wherein the comonomer is selected from the group consisting of perfluoroolefins and perfluoro(alkyl vinyl ethers).

40. A sensor array as set forth in claim 32, wherein the amorphous fluoropolymer comprises at least one selected from the group consisting of amorphous homopolymers and copolymers containing repeating cyclic structures formed during the cyclic polymerization of perfluoro(butenyl vinyl ether).
